(19) United States
(12) Patent Application Publication
(10) Pub. No.: US 2017/0042764 A1
(43) Pub. Date: Feb. 16, 2017

(54) Acupressure Device and Method of Treatment

(71) Applicants: Alex Tima, Montrose, CA (US); Kevin SooHoo, Montebello, CA (US); Joseph Hsiao-Hsien Chen, Los Angeles, CA (US)

(72) Inventors: Alex Tima, Montrose, CA (US); Kevin SooHoo, Montebello, CA (US); Joseph Hsiao-Hsien Chen, Los Angeles, CA (US)

(21) Appl. No.: 14/822,894

(22) Filed: Aug. 10, 2015

(51) Int. Cl. A61H 39/04; A61H 1/00; A61N 1/00

(52) U.S. Cl. CPC A61H 39/04; A61N 1/00; A61H 1/008

(57) ABSTRACT

An acupressure device and methods for application of the acupressure device to appendages of the human body for therapy and/or the treatment of various ailments.

### FIELD OF THE INVENTION

The invention relates generally to an acupressure device and methods for application of the acupressure device to appendages of the human body for therapy and/or the treatment of various ailments.

Complementary and alternative medicine practices have become widely used and accepted in the Western world as supplements and/or alternatives to conventional healthcare and medical methodology. Many alternative medical practices utilize natural therapies and/or ancient techniques such as acupuncture, acupressure, and magnet therapy. Although the actual medical benefits of acupuncture, acupressure, or magnet therapy have not yet been quantified by Western doctors or scientists, many people report significant improvement in their medical conditions as a result of these methods.

Acupressure has long been recognized as an effective treatment method for relieving ailments affecting the human body. The application of non-invasive pressure at specific points on the human body, particularly at recognized acupuncture points, can relieve pain and/or discomfort locally and at various other locations throughout the body. It is believed that this relief is obtained through the same mechanism of action as acupuncture. The application of pressure at these points may stimulate the body to release endorphins, enkephalins, inflammatory mediators, and other hormones, which can alleviate ailments such as pain, muscle stiffness, spasms, tension, sleep disorders, emotional disturbances, and various other conditions.

To assist practitioners and patients in employing acupressure, multiple devices have been contemplated to apply and sustain a fixed level of pressure at discrete acupressure points for the treatment of various conditions. Given that the human body comes in a variety of different shapes and sizes, with each patient having varying amounts of muscle, fat and other tissues, the application of a fixed level of pressure exerted by such a device may elicit the desired effect for one patient, while causing intolerable pain for another patient. Furthermore, the level of pressure necessary for effective treatment at one pressure point may vary compared to the effective pressure necessary for treatment at a second or third pressure point on the same patient.

As pressure is defined as force over the area upon which the force is applied, the contact point for applying the force to the patient is directly related to the effectiveness of the treatment for a particular ailment. The current state of the art teaches proper techniques for acupressure incorporating the use of multiple acupressure devices, each capable of applying a specific degree of pressure.

The present invention provides an advantageous method and device for allowing practitioners and self-treating patients to practice acupressure therapy. By providing a single device capable of finite adjustment of pressure, both by altering the contact point applied to the patient and by adjusting the amount of force exerted on the patient by the device, the present invention teaches a new device and method for effective practice of acupressure therapy.

Furthermore, the low cost and ease of use associated with the present invention allows for practice of acupressure therapy by an end-user patient, as well as extended acupressure therapy by the patient outside a physician's care.

### BRIEF DESCRIPTION OF THE DRAWINGS

These and other features of this invention will be more readily understood from the following detailed description of the various aspects of the invention taken in conjunction with the accompanying drawings in which:

FIG. 1 illustrates a side view of the acupressure device in the open configuration in accordance with an embodiment or portion of an embodiment of the present invention.

FIG. 2 depicts a side view of the acupressure device in the closed configuration in accordance with an embodiment or portion of an embodiment of the present invention.

FIG. 3 illustrates a perspective view of the lower arm of the acupressure device in accordance with an embodiment or portion of an embodiment of the present invention.

FIG. 4 depicts a perspective view of the upper arm of the acupressure device in accordance with an embodiment or portion of an embodiment of the present invention.

FIG. 5 illustrates a top perspective view of the acupressure device in the closed configuration in accordance with an embodiment or portion of an embodiment of the present invention.

FIG. 6 depicts a sampling of removable tips of the acupressure device in accordance with an embodiment or portion of an embodiment of the present invention.

FIG. 7 illustrates acupressure points identified on the human hand, and the treatment area associated with each acupressure point, in accordance with an embodiment or portion of an embodiment of the present invention.

FIG. 8 provides a listing of acupressure points identified throughout the human body, and the treatment area associated with each acupressure point, in accordance with an embodiment or portion of an embodiment of the present invention.

The attached drawings are merely schematic representations, and are not intended to portray specific parameters of the invention. Furthermore, the attached drawings are intended to depict only typical embodiments of the invention, and therefore should not be considered as limiting the scope of the invention. In the attached drawings, like numbering represents like elements.

### DESCRIPTION OF THE INVENTION

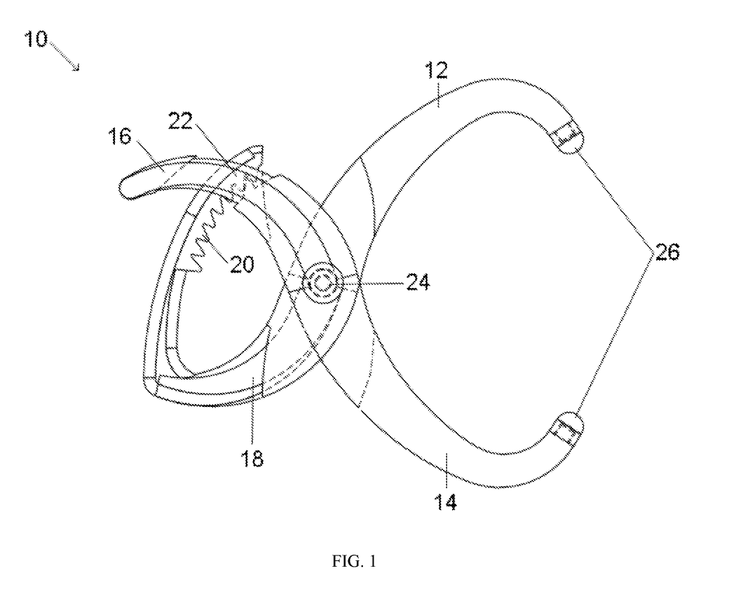

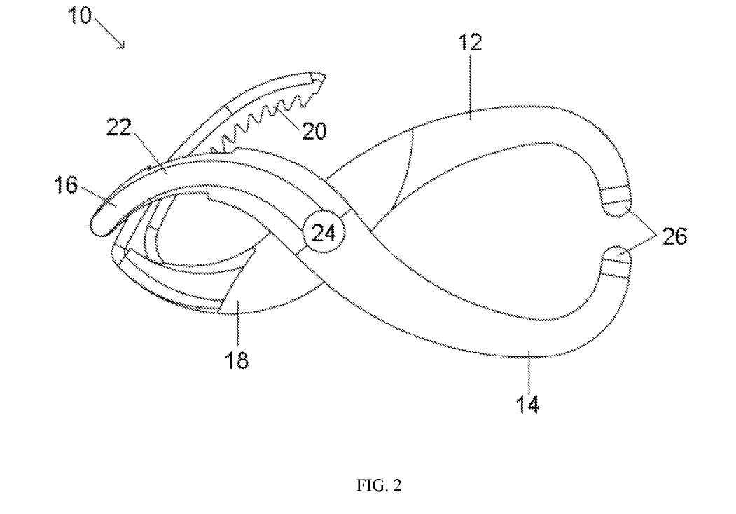

FIG. 1 illustrates a side view of the acupressure device in the open configuration in accordance with an embodiment or portion of an embodiment of the present invention. The acupressure device or acupressure clip 10 (also referred to as “clip”), comprises an upper arm 12 pivotally coupled to a lower arm 14, at a near central pivot point 24. As can be appreciated, the pivot point 24 may be contemplated in various positions to reach the desired movement ratio desired in the clip 10. In the present embodiment, a 1:1 ratio has been selected for exemplary purposes only. The upper arm 12 comprises a lower handle 18 at the distal end of the upper arm 12, which is designed to fit in the palm of the medical practitioner's or end user's hand. The lower arm 14 comprises an upper handle 16 at the distal end of the lower arm 14, which is designed to abut the practitioner's or end user's thumb. The lower arm 14 further comprises a pawl housing 22 configured adjacent to the upper handle 16 and the pivot point 24. The upper arm 12 further comprises a gear rack 20 configured adjacent to the lower handle 18 and the pivot point 24, whereas the gear rack 20 comprises at least two gears for engagement and locking of the upper arm 12 with respect to the lower arm 14, about the pivot point 24. The gear rack 20 is configured to be received in the pawl housing 22 and engages a pawl 28 found in the housing.

The proximal end of the upper arm 12 comprises one of two removable tips 26, configured to contact the end user and provide the therapeutic pressure. The second of the removable tips 26 is mounted to the proximal end of the lower arm 14. The removable tips 26 are configured to align with one another about the trajectory of the clip 10 when the upper arm 12 and lower arm 14 are moved towards one another along the pivot point 24. The tips 26 are removable by incorporating various fastening methods know in the industry, or combinations of fastening methods thereof, including compression fit, threaded fastener, button, cleko, hook-and-eye, pins, clips, snap fastener, variations thereof, and combinations therefor.

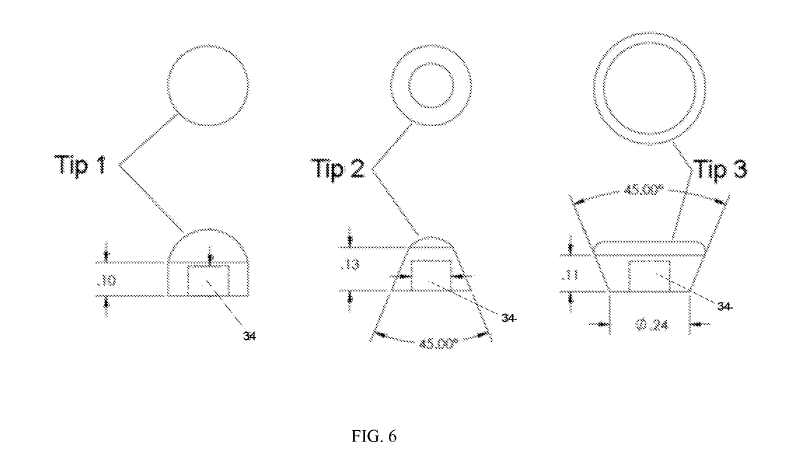

The dimensions of the clip 10 are approximately 3.2 inches in length; approximately 3.2 inches in height; with the width of the assembled clip being approximately 0.3 inches. The size of a removable tip 26 varies slightly from one tip type to another, and are approximately 0.3 in height×0.3 in length×0.3 in width, all in inches. (See FIG. 6)

The closed configuration of the clip 10, provided in FIG. 2, details interactions of the upper arm 12 with the lower arm 14 as the clip 10 is compressed. Of significance is the progression of the gear rack 20 through the pawl housing 22, which results in the removable tips 26 moving closer to one another (and if applied to an object, creating pressure). FIG. 2 also reveals the pawl release 30, which is used to disengage the gear rack 20 from the pawl 28, allowing the clip 10 to be re-positioned or moved into the open configuration (See FIG. 1). The present embodiment allows for complete disengagement of the gear rack 20 from the pawl housing 22, however, other embodiments may incorporate the use of a stop device for limiting disengagement of the gear rack 20 from the pawl housing 22. In yet another embodiment, the gear rack 20 and/or pawl housing 22 and/or pawl 28 may further incorporate a brake device for limiting the amount compression capable by the clip 10, in order to promote safety and/or functionality.

In yet another embodiment, the clip 10 may incorporate a measurement device for displaying the amount of force applied by the clip 10, when engaged on an acupressure point. Exemplary devices may include a torque arm/lever, pressure indicator, sensor, or other similar devices for measuring force.

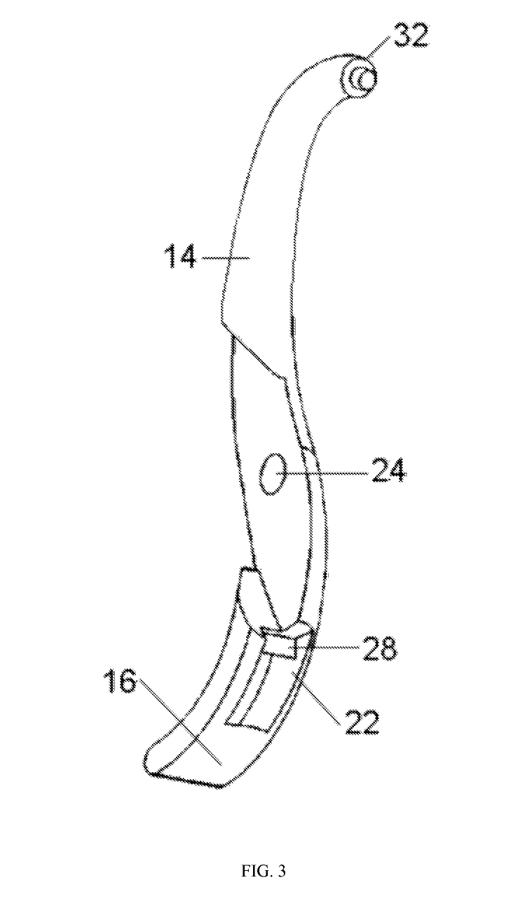

FIG. 3 provides a single element of the clip 10, namely, the lower arm 14. The lower arm 14 is shown disassembled from the clip 10, to better detail the functionality and interplay of the lower arm 14 with other components of the clip 10, namely, the upper arm 12 and gear rack 20. Furthermore, FIG. 3 provides a better illustration of the pawl 28, which engages with the gear rack 20 to lock the clip 10 in position. The pawl 28 is angled so as to complement the teeth found on the gear rack 20, allowing the clip 10 to functionally lock only when the clip 10 is compressed.

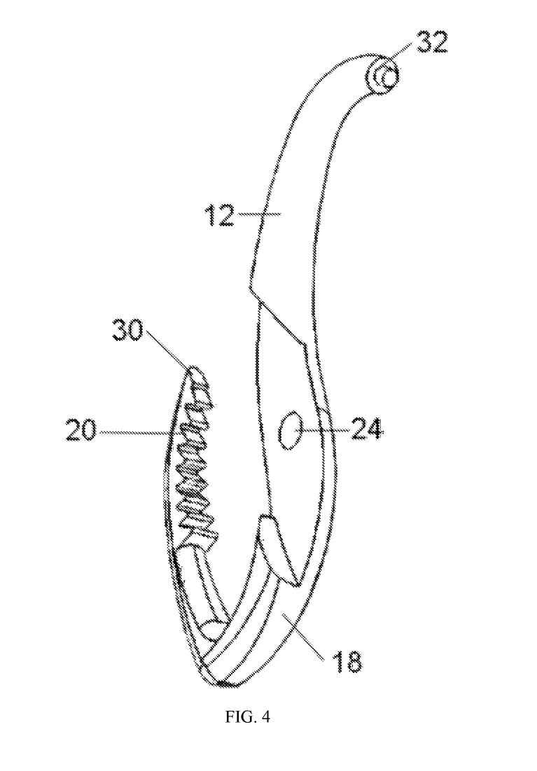

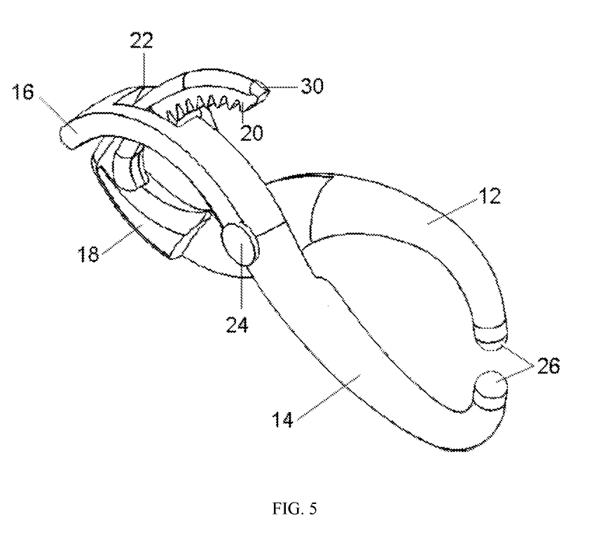

Similar to FIG. 3, FIG. 4 provides the disassembled upper arm 12 of the clip 10, detailing the gear rack 20, as well as the angles of the at least two teeth found on the gear rack 20. As stated in FIG. 3, the teeth of the gear rack 20 are angled to complement the pawl 28, allowing the clip 10 to functionally lock only when the clip 10 is compressed. Also depicted in FIG. 4 is the pawl release 30, configured at the end of the gear rack 20, for disengaging the gear rack 20 from the pawl 28, and releasing the clip 10.

FIG. 3 and FIG. 4 also depict a tip stud 32 found at the distal end of each arm (upper 12, lower 14) for engaging a removable tip 26. The depicted tip stud 32 is cylindrically shaped and designed to mate with a socket found on each removable tip 26, for compression fitting of the removable tip 26 to each arm 12 & 14. Although the present embodiment describes compression fitting for attachment of the removable tip 26 to the arms 12 & 14, the present invention hereby incorporates a host of alternative fastening means, including, but not limited to, threaded fastener, button, cleko, hook-and-eye, pins, clips, snap fastener, variations thereof, and combinations therefor.

FIG. 6 depicts a sampling of removable tips 26 for adaption to the clip 10. Identified in the illustration as Tip 1, Tip 2, and Tip 3, each removable tip 26 provides different contact area and engagement shapes. The illustrations provided in FIG. 6 show a top view and side view of each removable tip 26. Tip 1 is the most contemporary, providing a spherical contact area. Tip 2 is angled to provide a narrower contact area, leading to greater force than Tip 1. Tip 3 is angled to provide a wider contact area, leading to less force than both Tip 1 and Tip 2. All three removable tips 26 also depict a tip socket 34, for matting with the tip stud 32.

Although the present embodiment of the invention depicts removable tips 26 comprising rubber or synthetic materials, the present invention hereby contemplates the use of removable tips 26 comprising: precious metals (e.g. silver, copper or titanium), magnetic material, crystals (e.g. quartz, rose quartz, diamond or salts), herbs (e.g. A Ye—Artemisiae argyi Folium—a.k.a. mugwort, San Qi—Notoginseng Radix—a.k.a. notoginseng, and Bai Fan Alumen—a.k.a. alum), derivatives thereof, and combinations therefrom (Including, but not limited to Shao Yao Gan Cao Tang Peony and Licorice Decoction, Chuan Xiong Cha Tiao San—Chuanxiong Powder to be Taken with Green Tea, and Juan Bi Tang—Remove Painful Obstruction Decoction).

In various embodiments., the tips may be comprised of rubber or synthetic material with the desired one or more crystals, herbs, derivatives thereof and combinations therefrom, infused in the rubber or synthetic material. In yet another embodiment, the rubber or synthetic material may have a cavity for adding or replenishing one or more crystals, herbs, derivatives thereof and combinations therefrom.

It is further contemplated that the tips may incorporate a charge element, preferably battery operated, for imparting an electronic charge to the patient. The charge element may be configured to an appropriate charge gradient, as dictated in the industry.

It is further contemplated that the tips 26 may be configured to incorporate, or be in communication with, an electric motor. The electric motor is configured to oscillate the tip at a wide range of frequencies and amplitudes, creating resonance, vibration, and other physical movement of the tip 26.

Additional contemplated herbs, crystals and precious metals are hereby incorporated by reference to the following books, teachings and literary materials.
Chinese Acupuncture and Moxbiustion by Cheng Xinnon (ISBN-13: 978-7119059945)
Chinese Herbal Medicine: Materia Medica by Dan Bensky (ISBN-13: 978-0939616428)
Chinese Herbal Medicine: Formulas & Strategies by Volker Scheid & Dan Bensky (ISBN-13: 978-0939616671)
Chinese Medical Herbology & Pharmacology by John Chen (ISBN-13: 978-0974063508)
Chinese Herbal Formulas and Applications by John Chen (ISBN-13: 978-0974063577)

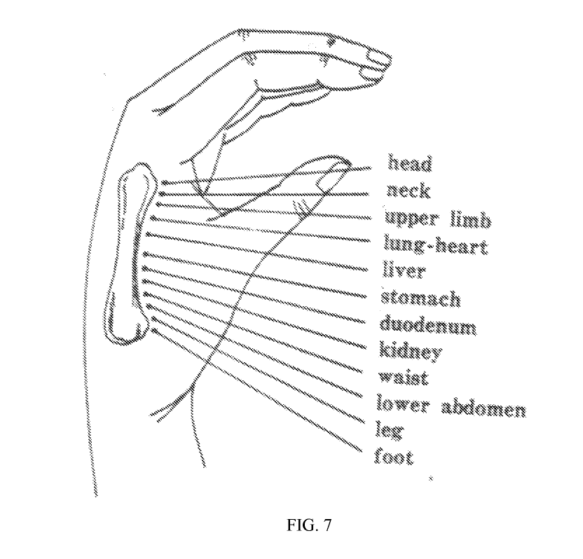

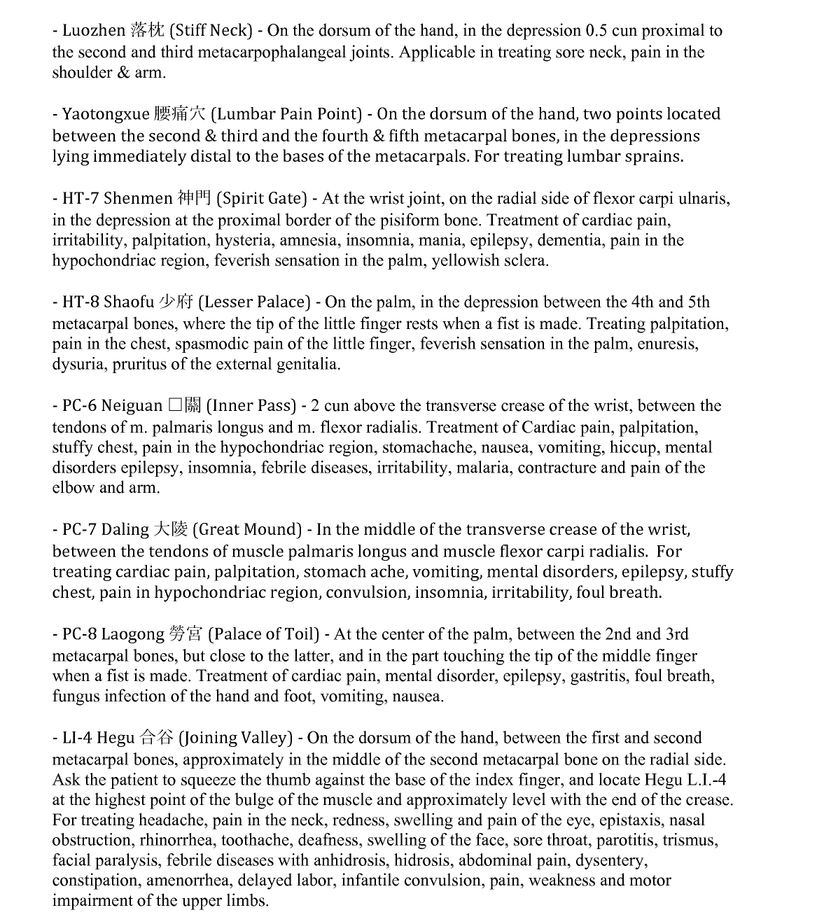

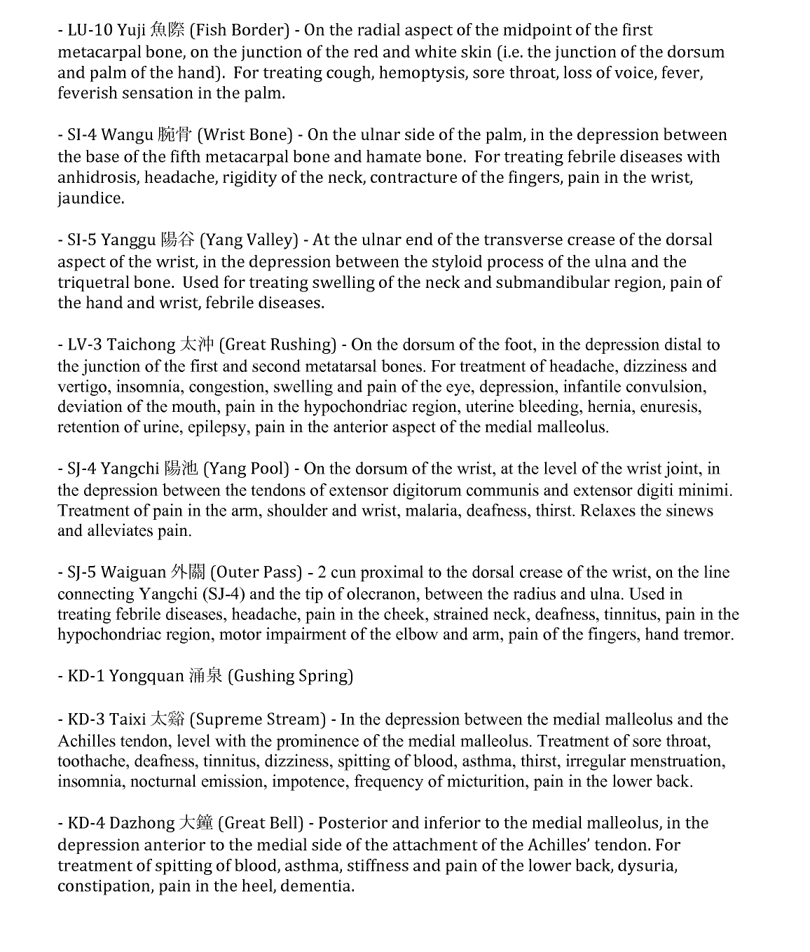

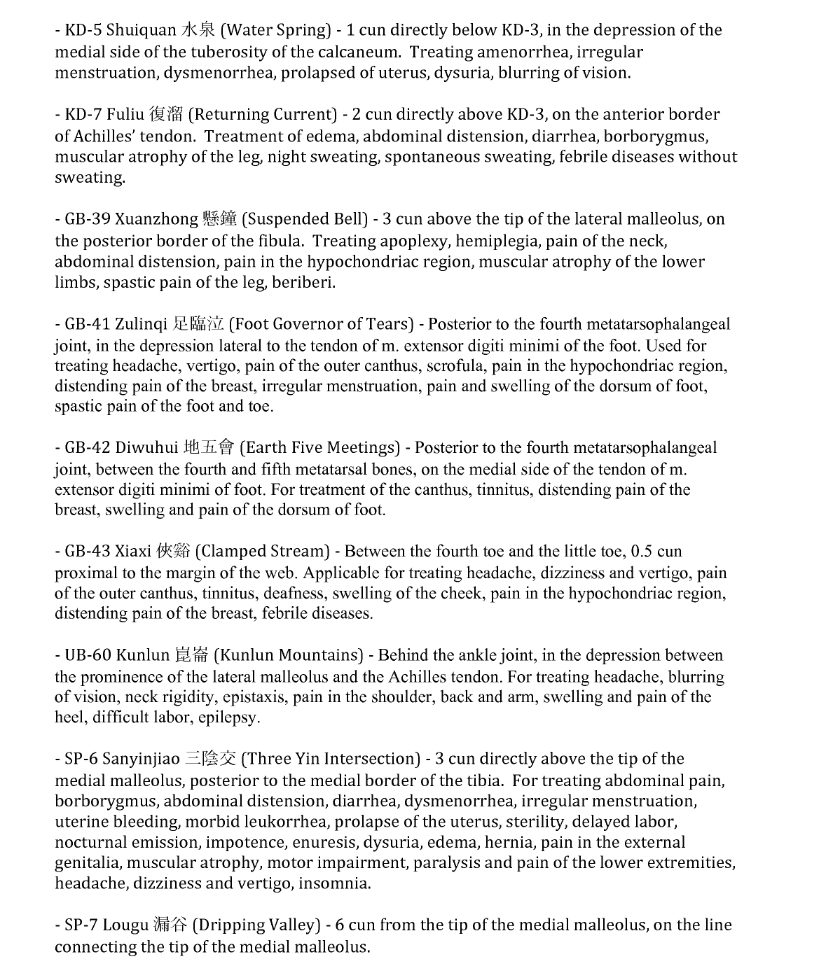

FIG. 7 provides acupressure points identified on the human hand, and the treatment area associated with each acupressure point. These acupressure points and others are hereby incorporated by reference to the following books, teachings and literary materials.
Chinese Acupuncture and Moxbiustion by Cheng Xinnong (ISBN-13: 978-7119059945)
Foundations of Chinese Medicine by Giovanni Maciocia (ISBN-13: 978-0443074899)
A Manual of Acupuncture by Peter Deadman (ISBN-13: 978-095105465)
Acupuncture: A Comprehensive Text by John O'Connor/Dan Bensky (ISBN 978-0-939616008)
Eciwo and Its Application to Medicine by Zhang Yingqing (ISBN-13: 978-7533109882)
ECIWO Biology and Bio-Holographic Acupuncture by Vilhelm Schjelderup (Acupuncture in Medicine, May 1992, Vol. 10, No. 1, ppgs 29-31.

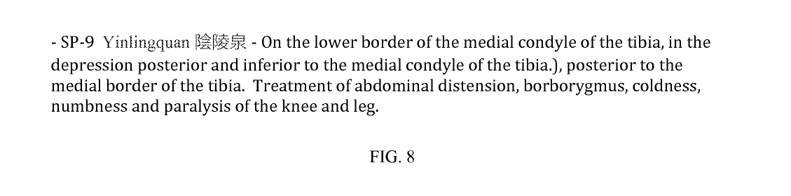

FIG. 8 provides a listing of acupressure points identified throughout the human body, and the treatment area associated with each acupressure point. In addition, Chinese characters have been provided for easier identification of each acupressure point.

In various physical therapies dealing with injured, aching, and tense muscles, electrical pulse stimulation has been applied and proven to be effective at providing comfort and relieving certain ailments. Electric pulse stimulation applies current across two ends of an affected muscle and/or associated nerves, to involuntarily contract (pulse-on) and relax (pulse-off) muscles, and to stimulate nerves to reduce pain, promote healing, and restore function. These treatments are called Electrical Muscle Stimulation (EMS) and Transcutaneous Electrical Nerve Stimulation (TENS). The conventional EMS and TENS used for therapy generate a train of electronic pulses with constant frequency, amplitude, and wave shape, which are chosen and set from a range of available options selected by the practitioner or the end user.

The present invention is capable of accepting electrical stimulus for transmission to the end user's body through the acupressure clip 10. Electrical stimulus may be accomplished by utilizing clip leads, plugs, and/or similar conductive means.

In practice, the compression clip 10, is fitted with the appropriate tip 26 required for the desired therapy, and the practitioner or end user places the lower portion of the clip 10 in his/her palm with his/her thumb resting on the upper handle 16. When the desired body region for acupressure is selected, the tips 26 are aligned with the body region and the clip 10 is compressed between said user's palm and thumb. The gear rack 20 ratchets into the pawl housing 22, compressing the clip 10 and locking the tips 26 on the desired body region with the desired pressure. Upon completion of the treatment, the clip 10 is released by disengaging the gear rack 20 from the pawl 28, thus releasing pressure on the body region applied by the tips 26.

In various embodiments, the gear rack 20 and pawl housing 22 assembly may be substituted with alternative fastening means, including, but not limited to, a strap, hook and loop fasteners.

Obviously, numerous additional modifications and variations of the present invention are possible in light of the above teachings. It is therefore to be understood that within the scope of the appended claims, the present invention may be practiced otherwise than as specifically described herein.

1. An acupressure tool, comprising:
an upper arm,
a lower arm coupled to the upper arm about the center of the upper arm and the center of the lower arm,
at least one tip configured at the end of each said upper arm and lower arm; and
a locking mechanism engaging the upper arm with the lower arm,
wherein,
the upper arm and lower arm rotate about the pivot point to move each of the at least one tips proximal and distal to one another.

2. The acupressure tool of claim 1, wherein the locking mechanism is releasable.

3. The acupressure tool of claim 1, wherein the tip is selected from a group consisting of a synthetic, rubber, derivative thereof and combinations therefrom.

4. The acupressure tool of claim 1, wherein the tip is selected from a group comprising of a precious metal, a magnetic material, a crystal, an herb, derivative thereof, and combinations therefrom.

5. The acupressure tool of claim 1, wherein the at least one tip is removably attached to the upper arm or lower arm.

6. The acupressure tool of claim 1, wherein the tip shape may be configured and selected from a group comprising of a cone, semi-circle, oval, cylinder, derivatives thereof, and combinations therefrom.

7. The acupressure tool of claim 1, further comprising a charge element in communication with the at least one tip, for imparting an electronic charge to the patient.

8. The acupressure tool of claim 1, further comprising an electric motor in communication with the at least one tip, for creating an oscillation onto the patient.

9. The acupressure tool of claim 8, wherein the oscillation is configured to create a resonance, a vibration, or other physical movement.

10. A method for providing acupressure therapy, comprising:
applying an acupressure tool to a patient,
wherein the acupressure tool comprises:
an upper arm,
a lower arm coupled to the upper arm about the center of the upper arm and the center of the lower arm,
at least one tip configured at the end of each said upper arm and lower arm; and
a locking mechanism engaging the upper arm with the lower arm,
wherein,
the upper arm and lower arm rotate about the pivot point to move each of the at least one tips proximal and distal to one another.

11. The method of claim 10, wherein the locking mechanism is releasable.

12. The method of claim 10, wherein the tip is selected from a group consisting of a synthetic, rubber, derivative thereof and combinations therefrom.

13. The method of claim 12, wherein the tip is selected from a group comprising of a precious metal, a magnetic material, a crystal, an herb derivatives thereof, and combinations therefrom.

14. The method of claim 10, wherein the at least one tip is removably attached to the upper arm or lower arm.

15. The method of claim 10, wherein the tip shape may be configured and selected from a group comprising of a cone, semi-circle, oval, cylinder, derivatives thereof, and combinations therefrom.

16. The method of claim 10, further comprising a charge element in communication with the at least one tip, for imparting an electronic charge to the patient.

17. The method of claim 10, further composing an electric motor in communication with the at least one tip, for creating an oscillation onto the patient.

18. The method of claim 17, wherein the oscillation is configured to create a resonance, a vibration, or other physical movement.
